(19) United States
(12) Patent Application Publication
(10) Pub. No.: US 2022/0248707 A1
(43) Pub. Date: Aug. 11, 2022

(54) CHEWING GUM COMPOSITIONS FOR THE TREATMENT OF MENOPAUSAL SYMPTOMS

(71) Applicant: ADVANCED FEMALE TECHNOLOGIES LLC, New York, NY (US)

(72) Inventor: Albert Feinstein, New York, NY (US)

(21) Appl. No.: 17/627,475

(22) PCT Filed: Jul. 16, 2020
(86) PCT No.: PCT/US2020/042362

§ 371 Date: Jan. 14, 2022

Related U.S. Application Data
(60) Provisional application No. 62/874,762, filed on Jul. 16, 2019.
(60) Provisional application No. 62/887,993, filed on Aug. 16, 2019.

(51) Int. Cl. A23G 4/06; A23G 4/08; A23G 4/10; A23L 33/105; A61K 36/48; A61K 45/06; A61K 9/68

(52) U.S. Cl. CPC A23G 4/068; A23G 4/08; A23G 4/10; A61K 9/0058; A61K 36/48; A61K 45/06; A23L 33/105

(57) ABSTRACT

This disclosure provides a chewing gum composition comprising an herbal extract comprising isoflavones and a gum base. The disclosure also provides methods of treating symptoms associated with menopause comprising administering to a subject in need thereof, a gum-based composition for chewing, the composition comprising an herbal extract comprising isoflavones and a gum base.

### CROSS-REFERENCE TO RELATED APPLICATIONS

This application claims the benefit of U.S. Provisional Application No. 62/874,762, filed Jul. 16, 2019, and U.S. Provisional Application No. 62/887,993, filed Aug. 16, 2019, both of which are incorporated herein by reference in their entirety.

### FIELD OF THE INVENTION

This disclosure provides chewing gum compositions comprising an herbal extract comprising isoflavones, e.g., from a red clover extract, and methods of treating symptoms associated with menopause.

### BACKGROUND

During menopause, a decrease in estradiol (E2) production accompanies menopause, as the ovaries cease manufacture of E2. This decrease in E2 production results in a shift in hormone balance in the body, which often gives rise to a variety of symptoms associated with menopause. Deficiency of estrogens during menopause can lead to a number of menopause-associated complications including hot flushes, reduced bone density, and mood swings. These symptoms are commonly treated with synthetic hormones.

Menopausal symptoms are known and are described, for example, by Greene, J. G. and Cooke, D. J. (1980) British Journal of Psychiatry Volume 136, 486-491 (incorporated herein by reference). Some of the major symptoms of menopause are, for example, hot flashes, sweating at night, heart beating quickly or strongly, feelings of tension or nervousness, difficulty in sleeping, excitability, attacks of panic, difficulties in concentrating, feelings of tiredness or lack of energy, unhappiness or depression, crying spells, irritability, feelings of dizziness or faintness, pressure or tightness in head or body, parts of the body feeling numb or tingling, dry vagina and/or dry mouth, headaches, muscle and joint pains, loss of feeling in hands or feet, breathing difficulties, and loss of interest in sex. Hot flashes and night sweats are two of the principal menopausal symptoms, which are uncomfortable for women experiencing menopause.

Phytoestrogens are a type of estrogen occurring naturally in legumes, and considered beneficial in some diets. Phytoestrogens and their metabolites possess weak estrogenic activity and compete for estrogen receptors in target tissues, including the central nervous system (hypothalamus/pituitary), uterus, breast cells, osteoblasts/osteoclasts, etc. Despite this weak intrinsic estrogenicity, the phytoestrogens may actually exert an attenuating antiestrogenic effect. See Rose, D. P., Nutrition, 8:47 to 51 (1992).

Red clover (Trifolium pratense) is a perennial herb traditionally used to treat skin disorders such as psoriasis and eczema, whooping cough, and mastitis. It contains compounds known as isoflavones that act as phytoestrogens. Red clover extract, is typically administered in the form of tablets or capsules. However such orally administered dosage forms are unable to provide fast relief for symptoms of menopause such as night sweats and hot flashes. There is a need in the art for a composition comprising red clover extract that can be used to treat or reduce the symptoms of menopause such as hot flashes where the potency and absorption of the active ingredient is enhance as compared to conventional ingestible orally-delivered products such as capsules, pills or liquid.

### SUMMARY OF THE INVENTION

In one aspect, the present disclosure provides a chewing gum composition comprising a red clover extract comprising isoflavones and a gum base. In embodiments, the gum base comprises one or more elastomers, resins, softening agents, emulsifiers and/or fillers.

In embodiments, the red clover extract comprises about 20% to about 60% isoflavones, about 30% to about 50% isoflavones, about 40% to about 45% isoflavones, or about 40% isoflavones.

In embodiments, the isoflavones include biochanin A, daidzein, formononetin, genistein and puerarin. In embodiments, the isoflavones comprise about 15% to about 35% biochanin; about 0.5% to about 4% daidzein; about 5% to about 20% formononetin; about 0.5% to about 10% genistein; and about 0.1% to about 3% puerarin. In embodiments, the isoflavones comprise about 22% to about 26% biochanin; about 1.5% to about 2.5% daidzein; about 12% to about 16% formononetin; about 2% to about 4% genistein; and about 0.8% to about 1.5% puerarin. In embodiments, the isoflavones comprise about 24% biochanin, about 2% daidzein, about 14% formononetin, about 3% genistein, and about 1% puerarin.

In embodiments, the gum base comprises couma macrocarpa, glycerol esters of gum, microcrystalline wax, lecithin and calcium carbonate.

In embodiments, the chewing gum composition further comprises one or more sugar alcohols. In further embodiments, the sugar alcohol is, or includes, maltitol, sorbitol, isomalt, and/or xylitol. The chewing gum compositions may also contain one or more additional sweeteners. In embodiments, the additional sweeteners are selected from a sugar, a high intensity sweetener, a sugar substitute, or a combination thereof. In a further embodiment, the sugar substitute is Stevia.

The chewing gum compositions may also contain one or more flavoring agents, and one or more lubricants and powder flow agents. In embodiments, the flavoring agent comprises one or more a mint flavoring agents including, e.g., peppermint oil, oil of wintergreen and/or spearmint oil. In embodiments, the lubricants and powder flow agents are magnesium stearate, and silicon dioxide.

In embodiments, the chewing gum composition comprises one or more sugar alcohols, a gum base, one or more flavoring agents, one or more sweeteners, an herbal extract comprising isoflavones, and tableting excipients. In further embodiments, the composition comprises about 45-65% sugar alcohols, about 15-35% gum base, about 2-15% flavoring agents and/or sweeteners, about 2-10% herbal extract and about 1-10% tableting excipients. In embodiments, the composition comprises about 55% sugar alcohols, about 27% gum base, about 9% flavoring agents and/or sweeteners, about 5% herbal extract and about 3% tableting excipients.

Thus, in embodiments, the chewing gum composition, in addition to the herbal extract comprising isoflavones and the gum base, comprises maltitol, sorbitol, isomalt, xylitol, Stevia, a flavoring agent, magnesium stearate, and silicon dioxide. In embodiments, the herbal extract is selected from one of more of red clover extract, black cohosh extract, dong quai extract, vitex berry extract, wild yam extract, soy extract and/or kudzu root extract. In embodiments, the herbal extract comprising isoflavones is red clover extract. In embodiments, the chewing gum composition comprises, consists essentially of, or consists of one, two, three, four or more isoflavones found in, or derived from, red clover extract. In further embodiments, the chewing gum composition may include one or more of chromium picolinate, calcium disuccinate, ammonium succinate, zinc difumarate hydrate, tocopherol acetate, glycine, triterpene glycosides, folic acid, vitamin B12, vitamin E, vitamin B6, riboflavin and selenium.

In embodiments, the above chewing gum compositions further comprise a cannabis extract. In embodiments, the chewing gum compositions further comprise one or more cannabinoids. In embodiments, the one or more cannabinoids includes, but are not limited to, one or more of cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM) and combinations thereof. In embodiments, the one or more cannabinoids is or comprises CBD. In embodiments, the one or more cannabinoids does not include CBD, or includes less than 5%, less than 2%, less than 1%, or less than 0.5% CBD.

In another aspect, the present disclosure provides a method of treating symptoms associated with menopause comprising administering to a subject in need thereof, a gum-based composition for chewing as described in the previous paragraphs and/or as described herein. In embodiments, the one or more symptoms associated with menopause are selected from night sweats, hot flashes, chills, sleep disturbances, insomnia, fatigue, irritability and/or inability to concentrate. In a preferred embodiment, the one or more symptoms associated with menopause are hot flashes and/or night sweats. In one embodiment, the subject is either peri-menopausal, menopausal or post-menopausal.

In embodiments, the chewing gum compositions comprising isoflavones described herein may be manufactured by a method comprising the steps of:
(i) placing a gum base in a container with a sugar alcohol and heating to an internally-measured temperature of between 140-160° F. to melt the gum base;
(ii) combining the herbal extract comprising isoflavones, sweeteners and flavoring agents, and mixing;
(iii) adding the melted gum base mixture to the mixture from step (ii) and mixing;
(iv) conditioning the mixture from step (iii) for at least about 6 hours at a temperature less than or equal to 75° F. and a relative humidity of 60%;
(v) milling to form particulates;
(vi) mixing the particulates of step (v) with a lubricant; and
(vii) tableting the mixture from step (vi).

### DETAILED DESCRIPTION OF THE INVENTION

The present disclosure provides a chewing gum composition comprising a gum base and an herbal extract comprising isoflavones. The chewing gum composition described herein provides faster release of isoflavones as compared to other red clover compositions, and is effective for the treatment of, or reduction of, symptoms associated with menopause, including night sweats and hot flashes. The chewing gum compositions disclosed herein provide onset of action in about 5 to about 10 minutes, or less, as compared to orally administered dosage forms like tablets and capsules, which can take up to 40 minutes to absorb through the digestive tract.

Due to the high density of blood vessels in the buccal mucosa, the chewing gum compositions described herein buccally deliver isoflavones resulting in direct access to the systemic circulation, avoiding first-pass hepatic metabolism, and avoiding exposure to the acidic environment of the gastrointestinal tract. In addition, the buccal mucosa has low enzymatic activity relative to the nasal and rectal routes. Thus, the potential for inactivation of buccally delivered isoflavones due to biochemical degradation is less rapid and extensive than other administration routes.

Herbal Extract

The chewing gum compositions disclosed herein comprise one or more herbal extracts that contain isoflavones. The term “herbal extract” as used herein is a botanical extract including extract of plant material where the plant is an herb, as well as other plants. The herbal extracts may be derived from any useful part of the plant, including but not limited to leaf, stem, bark, flower, fruit, seed, root, or the entire plaint, by any of several methods of extraction known in the art.

The term “extract” as used herein is a preparation containing one or more active ingredients (e.g., one or more isoflavones) derived from plant material. Extraction methods include, for example, cold extraction techniques, soxhlet extraction, decoction, supercritical extraction, microwave extraction, and/or extraction using one or more extraction solvents including, but not limited to, ethanol, methanol, acetone, acetonitrile, chloroform, water, hydroalcoholic mixture, and isopropyl alcohol.

Red Clover Extract

In embodiments, the compositions and methods described herein employ a therapeutically effective amount of a red clover extract containing isoflavones. Red clover, also known as Trifolium pretense, extract can be prepared from red clover, for example, by the following procedure. First, the raw red clover plant material is harvested and dried, for example, by sun-drying or applying heat. Next, the dried red clover material is minced, followed by extraction in an aqueous/organic solvent mixture. An aqueous phase is used to extract the water-soluble red clover isoflavones, while the organic solvent is used to extract the water-insoluble isoflavones. The organic solvent can be, for example, one or more of alcohol (e.g., ethanol), chloroform, acetone or ethyl acetate. The amount of solvent in the water can be between 0.1% and 99.9%, for example, about 60% ethanol in water.

The red clover isoflavones are extracted by exposing the red clover plant material to the water:solvent mix. The exposure time is indirectly proportional to the temperature of the mixture. The temperature of the mix can range between room temperature and boiling temperature. The exposure time can be between about 1 hour to 4 weeks. Times for optimal recovery of isoflavones include, for example, 2 weeks at 50° C. or 24 hours at 90° C. The supernatant is separated from the undissolved plant material, and the organic solvent removed by distillation. Finally, the aqueous supernatant is concentrated, usually, for example, by distillation.

In embodiments, the red clover extract comprises greater than 10%, greater than 20%, greater than 30%, or greater than 40% isoflavones. In embodiments, the red clover extract comprises about 20% to about 60%, about 30% to about 50%, or about 35% to about 45% isoflavones. In embodiments, the red clover extract comprises about 35%, about 40%, about 45% or about 50% isoflavones.

In embodiments, the chewing gum composition comprises, consists essentially of, or consists of one, two, three, four or more isoflavones found in, or derived from, red clover extract. In embodiments, the isoflavones found in, or derived from, the red clover extract include, but are not limited, to one or more of biochanin A, daidzein, formononetin, genistein and puerarin.

In embodiments, the composition of isoflavones in the red clover extract comprises about 15% to about 35% biochanin; about 0.5% to about 4% daidzein; about 5% to about 20% formononetin; about 0.5% to about 10% genistein; and about 0.1% to about 3% puerarin. In embodiments, the composition of isoflavones in the red clover extract comprises about 20% to about 30% biochanin; about 1% to about 3% daidzein; about 10% to about 18% formononetin; about 1% to about 5% genistein; and about 0.5% to about 2% puerarin. In embodiments, the composition of isoflavones in the red clover extract comprises about 22% to about 26% biochanin; about 1.5% to about 2.5% daidzein; about 12% to about 16% formononetin; about 2% to about 4% genistein; and about 0.8% to about 1.5% puerarin. In a further embodiment, the composition of isoflavones comprises about 24% biochanin, about 2% daidzein, about 14% formononetin, about 3% genistein, and about 1% puerarin.

Measurement of the isoflavone content in the red clover extract can be performed by methods known in the art. For example, the isoflavone content can be measured using HPLC by the methodology adapted from Griffith, A. P., Collison, M. W., (Journal of Chromatography A, (2001) 913:397-413). Briefly, the method utilizes acetonitrile extraction without acidification, with apigenin as the internal standard. Samples in acetonitrile/water are diluted to 50% acetonitrile, directly injected on a HPLC column (e.g., Symmetry C18), and column eluted with a gradient mobile phase of 95% water (pH 3.5 with H₃PO₄) and 5% acetonitrile, changing linearly in 30 min. to 30% water (pH 3.5 with H₃PO₄) and 70% acetonitrile. Detection can be accomplished, e.g., with a photodiode array HPLC detector, scanning 200-400 nm with quantification at 245 nm.

Kudzu Root Extract

Kudzu root, contains several isoflavone compounds, such as daidzin, puerarin, and daidzein. A variety of different procedures for isolating and purifying isoflavones from Kudzu root are known in the art. For example, Ohshima et al., (Planta Medica, 250-254 (1988), incorporated herein by reference) describes a method of extracting kudzu root, using acetone to first extract the non-glycosidic flavonoids. The dried root is then extracted with methanol, the methanolic extract is subsequently re-extracted with aqueous butanol, and the resulting butanol layer is passed over a chromatographic column, eluting with methanol. The resulting glycosidic fractions are further chromatographed over a silica gel column followed by two preparative high performance liquid chromatography columns.

Soy Extract

Soy extract contains the isoflavones, daidzin (1a), genistin (1b), glycitein (1c), 6″dadidzin-O-acetyl (1d), 6″-O-acetyl genistin (e), 6″-O-malonyl daidzin (1g), 6″-O-malonyl genistin (1h). Ohta et al., (Agric Biol. Chem., 43:7, 1415-1419 (1979)) describes a method of isolating and purifying isoflavones from soybeans in which defatted soybeans are extracted with ethanol and the resulting ethanol extracts are treated with acetone and ethyl acetate. The ethyl acetate extract is then fractionated over silica gel and Sephadex LH-20 columns followed by multiple recrystallizations. Farmakalidis et al., reported in the Journal of Agric. Food Chem., 33:385-389 (1985) the use of acetone mixed with 0.1 N HCl as an extraction solvent rather than ethanol in the Ohta et al. method. E. D. Walter (J. Amer. Chem. Soc., 63:3273-3276 (1941)) describes a method for the extraction and isolation of genistin and its aglycone, genistein, from soybeans, in which defatted soybean flakes having been extracted with hexane are twice extracted using methanol. Acetone is added to the combined methanolic extract to precipitate some of the phosphatides and other impurities. The supernatant is decanted and two volumes of water are added to precipitate out the genistin. Multiple recrystallizations are then performed to purify the genistin.

Black Cohosh Extract

Black cohosh (Actaea racemosa or Cimicifucra racemose) is a member of the buttercup family (Family Ranunculaceae). It grows in open woods and at the edges of dense forests from Ontario to Tennessee and as far west as Missouri. It is a tall, leafy, perennial herb with a long wand-like raceme of white flowers. The subterranean portion develops as a thick, knotted rhizome system. The roots/rhizomes have been used traditionally by Native Americans to treat colds, rheumatism, and a variety of conditions related to women's health. Black cohosh has traditionally been used to treat conditions including inflammation, dysmenorrhea, cough suppression, diarrhea, rheumatism, arthritis and muscle pain. It has also been used as a sedative and muscle relaxant.

Classes of compounds present in black cohosh extract include triterpene glycosides (actein, cimicifugoside, cimigoside, deoxyacetylacetol, racemoside and 23-epi-26-deoxyactein (previously known as 27-deoxyactein)), phenolic acids (hydroxycinnamic acids, caffeic acid, ferulic acid, and isoferulic acid) and alkaloids (cimipronidine, cyclocimipronidine, and dopargine), salicylic acid, tannins, resin, phytosterols, fatty acids starch and sugars. See Nikolić D., et al. (2015) Nitrogen-Containing Constituents of Black Cohosh: Chemistry, Structure Elucidation, and Biological Activities. In: Jetter R. (eds) The Formation, Structure and Activity of Phytochemicals. Recent Advances in Phytochemistry, vol. 45, Springer, and Black Cohosh Clinical Overview, ABC Clinical Guide to Herbs (2002), pp. 13-22, incorporated herein by reference. Black cohosh also contains isoflavones including formononetin.

Extract of black cohosh may be obtained from the dried rhizome and roots of the plant. Several preparations of black cohosh have been described. For example, a dried rhizome and root preparation was described by Bradley P (ed.). British Herbal Compendium Vol. 1. Exeter, UK: British Herbal Medicine Association; 1992:34-6. A decoction may be prepared by adding black cohosh (cut, dried root) to boiling water and simmering for 10-15 minutes. Other preparation include a fluid extract: 1:1 (g/ml) in 90% alcohol. Karnick C. Pharmacopoeial Standards of Herbal Plants. Delhi, India: Srit Satguru Publications; 1994; 1,2:61-2, 13; Newall C et al., Herbal Medicines: A Guide for Health-Care Professionals. London: The Pharmaceutical Press; 1996; Lust J. The Herb Book. New York, N.Y.: Bantam Books; 1974; 124-5, each incorporated by reference.

Wild Yam Extract

Wild yam refers to species in the genus Dioscorea (Dioscorea polystachya, Dioscorea oppositifolia, Discorea opposita thunb, Dioscorea villosa, Dioscorea alata). Wild yam contains compounds including flavonoids, steroidal saponins (dioscin and diosgenin), phytosterols (beta-sitosterol), alkaloids (dioscorin), tannins, starch, mucins, amylase, amino acids (arginine, glutamine, leucine, tyrosine), chlorine, calcium, chromium, copper, iron and vitamin C. The rhizome and root of wild yam may be extracted by methods known in the art.

Cannabis Extract

In embodiments, the chewing gum compositions disclosed herein comprise a cannabinoid plant extract. The cannabis plant has many naturally occurring substances that are of medical interest. Isolated compounds from the cannabis plant include 19-tetrahydrocannabinol (THC), cannabidiol (CBD), cannabichromene (CBC), cannabigerol (CBG), and cannabidivarin (CBDV), among other compounds. There are at least one hundred and forty one (141) cannabinoids that have been isolated from the cannabis plant.

Cannabinoids

Cannabinoids are a class of chemical compounds that acts on cannabinoid receptors, and include compounds found in cannabis, endogenous compounds, and synthetic cannabinoids. Examples of cannabinoids include, but are not limited to, cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM) and tetrahydrocannabinol (THC). Cannabinoids described in the present application include, but are not limited to, those listed in Table 1 below along with their standard abbreviations and chemical structure.

TABLE 1

| | | |
|---|---|---|
| CBD | Cannabidiol | 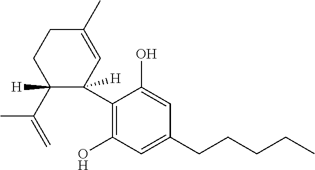 [SMILES: C=C(C)[C@@H]1CCC(C)=C[C@H]1c1c(O)cc(CCCCC)cc1O] |
| CBDA | Cannabidiolic acid | 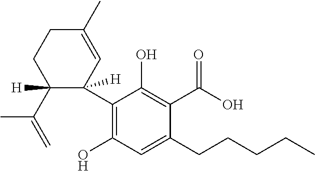 [SMILES: C=C(C)[C@@H]1CCC(C)=C[C@H]1c1c(O)cc(CCCCC)c(C(=O)O)c1O] |
| CBDV | Cannabidivarin | 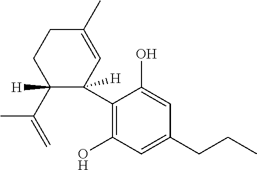 [SMILES: C=C(C)[C@@H]1CCC(C)=C[C@H]1c1c(O)cc(CCC)cc1O] |
| CBDVA | Cannabidivarinic acid | 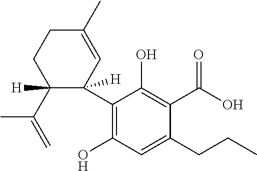 [SMILES: C=C(C)[C@@H]1CCC(C)=C[C@H]1c1c(O)cc(CCC)c(C(=O)O)c1O] |
| THC | Tetrahydrocannabinol | 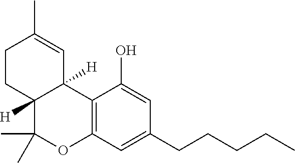 [SMILES: CCCCCc1cc(O)c2c(c1)OC(C)(C)[C@@H]1CCC(C)=C[C@@H]21] |

In embodiments, the chewing gum composition comprises CBD, another cannabinoid, combinations of CBD and other cannabinoids, or combinations of other cannabinoids. In embodiments, the CBD or another cannabinoid is in the form of a highly purified extract of cannabis such that the CBD or another cannabinoid is present at greater than 98% of the total extract (w/w). In embodiments, the cannabinoid tetrahydrocannabinol (THC) has been substantially removed, to a level of not more than 0.15% (w/w) and/or the propyl analogue of CBD, cannabidivarin, (CBDV) is present in amounts of up to 1%. In embodiments, the cannabis extract comprises less than about 0.10%, 0.05%, or 0.01% THC (w/w). In embodiments, the highly purified extract comprises no more than 0.15% CBDA and/or no more than 0.5% CBD-C4. Alternatively, the CBD may be a synthetically produced CBD.

In embodiments, the cannabinoid used in the chewing gum composition is obtained as a liquid carbon dioxide extract of high-CBD containing varieties of Cannabis sativa L. which had been further purified by a solvent crystallization method to yield CBD. The crystallization process specifically removes other cannabinoids and plant components to yield greater than 98% CBD. Although the CBD is highly purified, because it is produced from a cannabis plant rather than synthetically, there are other cannabinoids which are co-produced and co-extracted with the CBD. Similar methods for extracting cannabinoids other than CBD are also known in the art.

In embodiments, the chewing gum composition comprises about 2 to about 50 or more mg of a cannabis extract. In embodiments, the chewing gum composition comprises about 0.1 to about 50 or more mg of one or more cannabinoids. In embodiments, the chewing gum composition comprises about 0.1 to about 90 mg of CBD, or more, for example, about 0.1 mg to about 5 mg, about 1 mg to about 10 mg, about 5 mg to about 15 mg, about 10 mg to about 20 mg, about 15 mg to about 25 mg, about 20 mg to about 30 mg, about 25 mg to about 35 mg, about 30 mg to about 40 mg, about 35 mg to about 45 mg, about 40 mg to about 50 mg, about 45 mg to about 55 mg, about 50 mg to about 60 mg, about 55 mg to about 65 mg, about 60 mg to about 70 mg, about 65 mg to about 75 mg, about 70 mg to about 80 mg, about 75 mg to about 85 mg, or about 80 mg to about 90 mg, or more.

In embodiments, the chewing gum composition comprises CBD and THC. In such embodiments, the CBD is present in about 0.1-50 mg or more and the THC is present in about 0.1-50 mg or more, for example CBD and THC are independently present in about 0.1 mg to about 10 mg, about 1 mg to about 10 mg, about 5 mg to about 15 mg, about 10 mg to about 20 mg, about 15 mg to about 25 mg, about 20 mg to about 30 mg, about 25 mg to about 35 mg, about 30 mg to about 40 mg, about 35 mg to about 45 mg, or about 40 mg to about 50 mg.

Methods of Treating the Symptoms of Menopause

The present disclosure provides a chewing gum compositions for treating one or more symptoms associated with menopause, including symptoms associated with perimenopause. Treatment of menopausal symptoms is accomplished by administering a chewing gum composition disclosed herein to a subject in need thereof. The term treatment is used in its broadest sense and, as used herein, means lessening the severity, or otherwise alleviating menopausal symptoms. Administering as used herein means directing to take or taking (e.g., placing in the mouth and chewing) the chewing gum compositions described herein.

Embodiments provide a method of treating symptoms associated with menopause including hot flashes, sweating at night, rapid heartbeat, feelings of tension or nervousness, difficulty in sleeping, excitability, panic attacks, difficulties concentrating, feelings of tiredness or lack of energy, unhappiness or depression, crying spells, irritability, feelings of dizziness or faintness, pressure or tightness in head or body, numbness or tingling in body parts, dry vagina and/or dry mouth, headaches, muscle and joint pains, loss of feeling in hands or feet, breathing difficulties, and loss of interest in sex. Exemplary menopausal symptoms that can be treated by the gum compositions described herein include night sweats, hot flashes, headaches, chills, myalgia, sleep disturbances, dizziness, nausea, cold hands and feet, insomnia, fatigue, irritability and inability to concentrate. In preferred embodiments, the disclosure provides a method of treating hot flashes and night sweats associated with menopause.

In embodiments, the chewing gum for the treatment of menopausal symptoms comprises a therapeutically effective amount of red clover extract. In embodiments, the chewing gum composition comprises a therapeutically effective amount of one or more additional active ingredients or extracts as described herein. In embodiments, the chewing gum for the treatment of menopausal symptoms comprises a red clover extract which comprises about 40% of a combination of isoflavones. In other embodiments, the isoflavones are derived from additional or alternative sources of isoflavones including one or more of black cohosh extract, dong quai extract, vitex berry extract, wild yam extract, soy extract and kudzu root extract. During the chewing process, the isoflavones directly enter the blood stream through the oral mucosa (buccal or sublingual) allowing for fast alleviation of menopausal symptoms, such as hot flashes and night sweats. The absorption red clover extract through the oral mucosa is up to five times faster than conventional ingestible orally delivered products such as capsules, pills or liquids because it bypasses the digestive system and directly enters the bloodstream. Moreover, absorption of the isoflavones through the oral mucosa from the chewing gum compositions described herein, provides higher bioavailability of the isoflavones than isoflavones administered by conventional orally-delivered capsules, tablets, and liquids, in which the bioavailability is greatly reduced or absent.

The present disclosure provides a chewing gum delivery system that provides a therapeutically effective concentration of the isoflavones in the bloodstream within five minutes, and preferably within 1-2 minutes, of administration. In certain embodiments, the chewing gum composition has a high initial release rate of the red clover extract to provide fast relief of the symptoms of menopause.

The chewing gum compositions described herein provide red clover extract in a transmucosally absorbable form. In embodiments, absorption from the gum formulation occurs primarily through the buccal mucosa, which increases the rate of absorption of the active ingredients. Isoflavones administered in the chewing gum formulation are absorbed at a significantly faster rate, while bioavailability is comparable to, or greater than, that of the capsule formulation. As the onset of action for the gum delivery is within 5 to 10 minutes, or less, of administration, the dose of isoflavones can be quickly and easily titrated. Consequently the chewing gum composition described is a convenient and effective means of rapidly administering isoflavones, and is useful for the rapid alleviation of discomforting symptoms associated with menopause such as hot flashes and night sweats.

The method of treating the symptoms associated with menopause comprise chewing a piece of gum comprising the red clover extract as described herein, following the onset of a particular menopausal symptom, such as a hot flash. Typically, up to two pieces of the chewing gum composition may be chewed at one time. If symptoms associated with menopause continue after chewing of 1-2 pieces of gum, an additional piece of gum may be chewed. In embodiments, there is 40 mg of isoflavones in each piece of gum, and the suggested dosage for treating menopause symptoms is 120 mg per day.

The chewing gum compositions disclosed herein may be administered prophylactically to prevent one or more symptoms associated with menopause. The compositions disclosed herein may be administered alone or may be administered in combination with one or more other therapeutic agents.

Subject in Need Thereof

A subject in need thereof is any person that suffers from symptoms associated with a hormonal imbalance, including for example, menopause and/or perimenopause. The person can be in perimenopause (which is the time before the actual cessation of menstruation occurs, and can last for several years), menopause (having not had a menstrual period for 12 consecutive months), or may be post-menopausal (having not had a menstrual period for over two years).

Manufacture of the Gum Compositions

The chewing gum compositions described herein comprise a gum base and a red clover extract comprising isoflavones. In embodiments, methods for the preparation of the chewing gum composition comprising the red clover extract avoid heating and cooling of the active ingredient.

In an embodiment, the method for manufacture of the chewing gum is exemplified in U.S. Pat. No. 9,744,128, incorporated herein by reference. In general the process for preparing the chewing gum compositions comprises placing one or more gum base(s) in a container with a sugar alcohol and heating the gum base(s) and one or more sugar alcohols to an internally-measured temperature between 140-160° F. to melt the gum base(s). The gum base may contain one or more elastomers, resins, softening agents, emulsifiers and/or fillers. The ingredients, including one or more active ingredients, sugar alcohol(s) and flavorings are then combined and mixed in a commercial mixer. The melted gum base is added to the mixer and mixed until a homogenous mass is produced, and cooled to produce a particulate mixture. The temperature of the gum base initially exceeds that of the mixer when first introduced, but as mixing continues the mixture cools to room temperature and forms granular pieces. These granular pieces are then conditioned for a period of time, which allows the granular pieces to dry slightly and complete the crystallization process. The pieces are conditioned for at least about 6 hours at a temperature not greater than about 75° F. and about 60% or less relative humidity. The dried mass is then milled into particulates at room temperature using a mesh screen of appropriate size. The particulates are mixed with tableting excipients (e.g., one or more lubricants/glidants) in an orbital or planetary mixer, and tableted using a tablet press. This process preserves the efficacy of the active ingredient or ingredients by avoiding exposure to extreme temperatures, particularly during the mixing and milling.

In embodiments, other gum base compositions may be applied to the chewing gum compositions disclosed herein. The gum base can be prepared via application of traditional mixing that utilizes mechanical actions while heating the mixture.

In an embodiment, the mixing pot is preheated to approximately 115-125° C. After 10 minutes of preheating the elastomer, the filler and additional softening ingredients are added to the mixing pot. The rubber along with the resin are mixed until the mixture is consistent. The softening ingredients can be added after 30-50 minutes and mixed with the rubber and resin until a consistent mixture is achieved. The mixture can then be released into another pot to cool down to approximately 18-25° C.

In an embodiment, the mixing pot is preheated to approximately 55° C. After 10 minutes of preheating the gum base and filler are mixed in the mixing pot. After the mixture is consistent, additional ingredients are added. Herbal extract comprising isoflavones shall be added in the first half of the mixing process.

Chewing Gum Compositions

The present disclosure provides a chewing gum composition comprising a gum base and an herbal extract comprising isoflavones along with additional excipients. In embodiments, the herbal extract comprising isoflavones is red clover extract. In embodiments, the red clover extract comprises about 40% to about 45% isoflavones. In certain embodiments, the isoflavones also comprise soy isoflavones.

In embodiments, the composition comprises red clover extract and a gum base. The chewing gum compositions can additionally include a sugar, a sugar blend, a sugar alcohol, a blend of sugar alcohols or combinations thereof, as well as additional excipients such as coloring agents, flavoring agents, lubricants and powder flow agents. In further embodiments the composition comprises one or more sugar substitutes and flavoring agents. Preferred flavors for masking the taste of the red clover include mint, peppermint, citrus, mixed berries, spearmint, wintergreen, and combinations thereof.

In embodiments, the chewing gum composition comprises one or more sugar alcohols, a gum base, one or more flavoring agents, one or more sweeteners, an herbal extract comprising isoflavones, and tableting excipients. In further embodiments, the composition comprises about 45-65% sugar alcohols, about 15-35% gum base, about 2-15% flavoring agents and/or sweeteners, about 2-10% herbal extract comprising isoflavones and about 1-10% tableting excipients. In embodiments, the composition comprises about 55% sugar alcohols, about 27% gum base, about 9% flavoring agents and/or sweeteners, about 5% herbal extract and about 3% tableting excipients. In embodiments, the herbal extract comprising isoflavones is selected from one or more of red clover extract, black cohosh extract, dong quai extract, vitex berry, soy, kudzu root, and wild yam. In embodiments, the chewing gum composition comprises, consists essentially of, or consists of one, two, three, four or more isoflavones found in, or derived from, red clover extract.

In an embodiment, the chewing gum composition comprises about 5% to about 80% of a gum base; about 10% to about 80% by weight of a sugar, a sugar blend, sugar alcohol, blend of sugar alcohols, sweetener, or combinations thereof; about 1% to about 20% by weight of a flavoring agent; about 0.1% to about 10% by weight of a lubricant or powder flow agent, or combinations thereof. In an embodiment, the chewing gum composition comprises about 55% to about 75% of a sugar, a sugar blend, sugar alcohol, blend of sugar alcohols, sweetener, or combinations thereof, about 20% to about 30% of a gum base, about 1% to about 15% of a flavoring agent or agents, about 1% to about 20% of red clover extract comprising isoflavones, about 0.1% to about 5% tableting lubricants and powder flow agents; and about 0.01% to about 2% by weight of one or more sugar substitutes.

Gum Base

The gum base imparts the chewing characteristics to the final gum composition. The gum base can also impact the release profile of the active ingredients, flavors and sweeteners. A suitable chewing gum base for use in the gum compositions described herein comprises one or more constituents including one or more elastomer, resin, plasticizer or softener, and filler. Certain components in the gum base may have more than one function in the composition. The gum base may also contain one or more stabilizing agents, coloring agents, and flavoring agents. In a preferred embodiment, the gum base comprises couma macrocarpa, glycerol esters of gum, microcrystalline wax, lecithin, and calcium carbonate.

In embodiments, the chewing gum composition comprising the red clover extract comprises an elastomeric or natural chicle base as is commonly used in chewing gum formulations that are commercially available and accepted by the consumer. An elastomeric or natural chicle base is present in the chewing gum composition in an amount of about 10% to about 85% by weight, based on the total weight of the chewing gum composition.

Elastomers provide the rubbery, cohesive nature to the gum and may include natural or synthetic types. The elastomer may be any water-insoluble polymer including the natural and synthetic gum polymers utilized for chewing gums listed in the U.S. Code of Federal Regulations, Title 21, Section 172.615 (incorporated herein by reference). Useful natural elastomers include natural rubber such as smoked or liquid latex and guayule, and natural gums such as jelutong, couma macrocarpal (also called lechi caspi), perillo, sorva, massaranduba balata, massaranduba chocolate, nispero, rosidinha, chicle, gutta percha, gutta kataiu, niger gutta, tunu, chilte, chiquibul, and/or gutta hang kang.

Synthetic elastomers include high-molecular weight elastomers such as butadiene-styrene copolymers, polyisobutadiene and isobutylene-isoprene copolymers, low-molecular weight elastomers such as polybutene, polybutadiene and polyisobutylene, vinyl polymeric elastomers such as polyvinyl acetate, polyethylene, vinyl copolymeric elastomers such as vinyl acetate/vinyl laurate, vinyl acetate/vinyl stearate, ethylene/vinyl acetate, polyvinyl alcohol or mixtures thereof. Combinations of synthetic elastomer in the gum base can include a synthetic elastomer having a high-molecular weight and a low-molecular-weight elastomer. Combinations of synthetic elastomers include, but are not limited to, polyisobutylene and styrene-butadiene, polyisobutylene and polyisoprene, polyisobutylene and isobutylene-isoprene copolymer (butyl rubber) and a combination of polyisobutylene, styrene-butadiene copolymer and isobutylene isoprene copolymer, and all of the above individual synthetic polymers in admixture with polyvinyl acetate, vinyl acetate-vinyl laurate copolymers, respectively and mixtures thereof.

Resins provide a cohesive body or strength to the gum composition and may also act as softeners and include glycerol esters of gum, terpene resins, and/or polyvinyl acetate.

Plasticizers (softening agents) affect the firmness of the gum base. Elastomer plasticizers include natural rosin esters often referred to as ester gums. Such plasticizers include methyl, glycerol and pentaerythritol esters of rosins and modified rosins, such as hydrogenated, dimerized and polymerized rosins. Examples of plasticizers include glycerol ester of wood and gum rosin, glycerol ester of partially hydrogenated wood and gum rosin, glycerol ester of polymerized wood and gum rosin, glycerol ester of partially dimerized wood and gum rosin, glycerol ester of tall oil rosin, pentaerythritol ester of wood and gum rosin, pentaerythritol esters of partially and fully hydrogenated wood and gum rosin, methyl esters of wood and gum rosins and partially and fully hydrogenated methyl esters of wood and gum rosin. Synthetic plasticizers include terpene resins derived from α-pinene, β-pinene and/or dipentene.

Emulsifying agents may act as softeners and can also help to hydrate the composition. In embodiments, the emulsifier is lecithin and/or glycerol monostearate. Emulsifiers include monoglycerides, diglycerides, acetylated mono and diglycerides, distilled mono- and diglycerides, glycerol monostearate, propylene glycol monostearate, Na-, K-, Mg- and Ca-stearates, glycerol triacetate, fatty acid monoglycerides (e.g., stearic, palmitic, oleic and linoleic acids), lactic acid esters and acetic acid esters of mono- and diglycerides, sugar esters of edible fatty acids, lecithin and hydroxylated lecithin.

One or more fillers may be used in the compositions disclosed herein to modify the texture and aid in processing. In embodiments, the filler comprises talc and/or calcium carbonate. Fillers include celluloses and cellulose derivatives including microcrystalline cellulose, hydroxypropylcellulose and sodium carboxymethylcellulose, lactose, starches including potato starch and corn starch, carbohydrates including a cellulose derivative, e.g. hemicellulose. The cellulose derivative may be of natural origin, e.g. dextran, agarose, agar, pectin, alginate, xanthan, chitosan, starch. The cellulose derivative may also be of synthetic or semi-synthetic origin. In certain embodiments, the filler comprises microcrystalline cellulose (MCC), bamboo fibers, or combinations thereof. Specific examples of a suitable microcrystalline cellulose is microcrystalline cellulose comprising: AVICEL™ grades PH-100, PH-102, PH-103, PH-105, PH-112, PH-113, PH-200, PH-300, PH-302, VIVACEL™ grades 101, 102, 12, 20 and EMOCEL™ grades 50M and 90M, and the like, and mixtures thereof. The fillers may be present in the composition from about 5% to about 50% by weight, based on total weight of the composition.

Isoflavones

The present disclosure provides a composition for the treatment of symptoms associated with menopause comprising a gum base and at least one herbal extract comprising isoflavones. Isoflavones are a class of substituted derivatives of isoflavone, many of which may act as phytoestrogens in mammals. In embodiments, the isoflavones comprise one or more of genistein, daidzein, biochanin A, puerarin, glycitein or formononetin. The herbal extract comprising isoflavones is selected from one or more of red clover extract, black cohosh extract, dong quai extract, soy extract, kuzu root extract, wild yam extract and vitex berry extract. In embodiments, the isoflavones generally comprise about 20% to about 60% of the herbal extract, preferably about 30% to about 50% of the extract, and more preferably about 40% to about 50% the extract. In the case of red clover extract, the isoflavones comprise about 20% to about 60% of the red clover extract, preferably about 30% to about 50% of the red clover extract, more preferably about 40% to about 50% red clover extract.

Sweeteners

Sweeteners may be used in the compositions disclosed herein to impart a sweet taste to the gum composition. In addition, sweeteners can act a softener and/or as a filler in the gum composition. Suitable sweeteners include one or more of a sugar, sugar blend, sugar alcohol, a blend of sugar alcohols, high intensity sweeteners, sugar substitutes, or combinations thereof. Sugar substitutes include sucralose, monk fruit, neotame, licorice extract, stevia, honey or agave nectar, or combinations thereof. Suitable high intensity sweeteners include sucralose, stevia, honey, monk fruit, neotame, licorice extract, agave nectar, or combinations thereof. Sugar alcohols include sorbitol, isomalt, xylitol, maltitol, mannitol, erythritol or combinations thereof. Sugars include dextrose, sucrose, fructose, glucose or combinations thereof. The amount of sweetener in the gum composition depends on factors such as potency of the sweetener, rate of release, desired sweetness of the product, and amount and type of flavor used.

Flavorings

Natural and/or artificial flavorings may be used in the gum compositions disclosed herein. Both synthetic flavoring agents and natural flavoring agents derived from plants, leaves, flowers, fruits, etc. and combinations thereof can be used. Examples of flavoring agents include spearmint oil, cinnamon oil, oil of wintergreen (methysalicylate) and peppermint oils. Synthetic and natural fruit flavors useful as flavoring agents include citrus oil, e.g., lemon, orange, lime and grapefruit; fruit essences including apple, strawberry, cherry, banana, pineapple; and other flavorings such as aldehydes and esters including cinnamyl acetate, cinnamaldehyde, citral diethyl acetal, dihydrocavryl acetate, eugenyl formate, p-methylamisol, and others. Flavoring agents are generally liquids. However, they can also be used as spray dried solids. The use of flavoring agents having other physical forms such as powdered flavorings, beaded flavorings and encapsulated flavorings may also be used in the gum compositions disclosed herein. Preferred flavoring agents in the chewing gum compositions described herein include mint, peppermint, citrus, mixed berries, spearmint, wintergreen, and combinations thereof.

The amount of flavoring agent employed is determined by flavor type and the strength of flavor desired. In embodiments, flavoring amounts of about 0.05% to about 3.0% by weight of the overall chewing gum composition are used, preferably about 0.3% to about 1.5%, more preferably about 0.7% to about 1.2% by weight.

In embodiments, additional flavoring agents are added to the chewing gum composition granules immediately prior to tableting. Preferably, the additional flavoring agent is in a dry form, e.g., spray dried flavoring or encapsulated flavoring. However, liquid flavoring can be added if it is first blended with the dry components of the lubricating system.

Lubricants and Powder Flow Agents

Lubricants and powder flow agents (e.g., glidants) may be used in the compositions disclosed herein to aid processing. Tableting lubricants and powder flow agents include magnesium stearate, silicon dioxide, calcium stearate, stearic acid, talc, rice bran-based excipients, or combinations thereof.

Binders

Binders include water-soluble synthetic polymer, polyvinlypyrrolidone (povidone), sorbitol, mannitol, xylitol, lactitol, erythritol, pregelatinized starch, modified starch (e.g., starch sodium octenylsuccinate), hydroxypropylmethylcellulose and others.

Stabilizing Agents

In embodiments, the gum compositions contain one or more stabilizing agent to prolong the shelf life of the active ingredient(s) and/or other components of the gum composition. In embodiments, the stabilizing agent is an antioxidant. Suitable antioxidants include butylated hydroxyanisole (BHA), butylated hydroxytoluene (BHT), betacarotenes, tocopherols, acidulants such as vitamin C, propyl gallate, and combinations thereof.

All references referred to herein are incorporated in their entirety.

### EXAMPLES

Preparation of Red Clover Extract: Red clover is harvested and dried by sun-drying or applying heat. Next, the dried red clover material is triturated, followed by extraction in in an aqueous/organic solvent mixture containing 60% ethanol in water. The supernatant is separated from the undissolved plant material and the organic solvent removed by distillation. Finally, the aqueous supernatant is concentrated.

Preparation of the Chewing Gum Containing Red Clover Extract: In general the process for preparing the chewing gum compositions comprises placing the components of the gum base in a container with a sugar alcohol and heating in an oven to an internally-measured temperature between 140-160° F. to melt the gum base. The ingredients, including the active ingredient, sugar alcohol(s) and flavorings are then combined and mixed in a commercial mixer. The melted gum base is added to the mixer and mixed until a homogenous mass is produced, and cooled to produce a particulate mixture. The temperature of the gum base initially exceeds that of the mixer when first introduced, but as mixing continues the mixture cools to room temperature and forms granular pieces. These granular pieces are then conditioned for a period of time, which allows the granular pieces to dry slightly and complete the crystallization process. The pieces are conditioned for at least about 6 hours at a temperature not greater than about 75° F. and about 60% or less relative humidity. The dried mass is then milled into particulates at room temperature using a mesh screen of appropriate size. The particulates are mixed with tableting excipients in an orbital or planetary mixer, and tableted.

TABLE 2
Formulation of chewing gum

| Ingredient | wt % in gum | Function |
|---|---|---|
| Red clover extract | 5.4 | Active agent |
| (40% isoflavones) | | |
| Gum Base | 27.3 | elastomer |
| couma macrocarpa | | resin |
| glycerol ester of gum | | softening agent |
| microcrystalline wax | | emulsifier |
| lecithin | | filler |
| calcium carbonate | | |
| Sugar Alcohol | 55.5 | sweetener |
| maltitol | | |
| sorbitol | | |
| isomalt | | |
| xylitol | | |
| Flavoring agents and | 8.7 | flavoring agent, |
| sweeteners (stevia extract, | | sweetener |
| mint flavoring) | | |
| Tableting excipients (silicon | 3.1 | Glidant/lubricant |
| dioxide, Mg stearate) | | |

1. A composition comprising an herbal extract comprising isoflavones and a gum base.

2. The composition of claim 1, wherein the herbal extract is selected from one or more of red clover extract, black cohosh extract, dong quai extract, vitex berry extract, soy extract, kudzu root extract, and wild yam extract.

3. The composition of claim 1, wherein the herbal extract is red clover extract.

4. The composition of claim 3, wherein the red clover extract comprises about 20% to about 60% isoflavones.

5. The composition of claim 3, wherein the red clover extract comprises about 30% to about 50% isoflavones.

6. The composition of claim 3, wherein the red clover extract comprises about 40% isoflavones.

7. The composition according to claim 1, wherein the isoflavones comprise one or more of the group consisting of biochanin A, daidzein, formononetin, genistein, glycitein and puerarin.

8. The composition according to claim 1, wherein the isoflavones comprise biochanin A, daidzein, formononetin, genistein and puerarin.

9. The composition according to claim 1, wherein the isoflavones comprise about 15% to about 35% biochanin; about 0.5% to about 4% daidzein; about 5% to about 20% formononetin; about 0.5% to about 10% genistein; and about 0.1% to about 3% puerarin.

10. The composition according to claim 1, wherein the isoflavones comprise about 22% to about 26% biochanin; about 1.5% to about 2.5% daidzein; about 12% to about 16% formononetin; about 2% to about 4% genistein; and about 0.8% to about 1.5% puerarin.

11. The composition according to claim 1, wherein the isoflavones comprise about 24% biochanin, about 2% daidzein, about 14% formononetin, about 3% genistein, and about 1% puerarin.

12. The composition according to claim 1, wherein the gum base comprises one or more elastomers, resins, softening agents, emulsifiers and fillers.

13. The composition according to claim 1, wherein the gum base comprises couma macrocarpa, glycerol esters of gum, microcrystalline wax, lecithin and calcium carbonate.

14. The composition according to claim 1, further comprising one or more sugar alcohols.

15. The composition of claim 14, wherein the one or more sugar alcohols comprises one or more of maltitol, sorbitol, isomalt, and xylitol.

16. The composition according to claim 1, further comprising one or more additional sweeteners.

17. The composition of claim 16, wherein the one or more additional sweeteners are selected from a sugar, a high intensity sweetener, a sugar substitute, or a combination thereof.

18. The composition according to claim 1, further comprising one or more flavoring agents, and one or more lubricants and powder flow agents.

19. The composition according to claim 1, wherein the composition comprises about 45-65% sugar alcohols, about 15-35% gum base, about 2-15% flavoring agents and/or sweeteners, about 2-10% herbal extract comprising isoflavones, and about 1-10% tableting excipients.

20. The composition of claim 19, wherein the composition comprises about 55% sugar alcohols, about 27% gum base, about 9% flavoring agents and/or sweeteners, about 5% herbal extract and about 3% tableting excipients.

21. The composition according to claim 1, further comprising maltitol, sorbitol, isomalt, xylitol, Stevia, a flavoring agent, magnesium stearate, and silicon dioxide.

22. The composition according to claim 1, further comprising one or more of chromium picolinate, calcium disuccinate, ammonium succinate, zinc difumarate hydrate, tocopherol acetate, glycine, triterpene glycosides, folic acid, vitamin B12, vitamin E, vitamin B6, riboflavin and selenium.

23. The composition according to claim 1, further comprising one or more cannabinoids.

24. The composition of claim 23, wherein the one or more cannabinoids comprises a cannabinoid selected from the group consisting of cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM) and combinations thereof.

25. The composition of claim 24, wherein the one or more cannabinoids is CBD.

26. A method of treating one or more symptoms associated with menopause comprising: administering to a subject in need thereof, a composition according to claim 1.

27. The method of claim 26, wherein the one or more symptoms associated with menopause are selected from the group consisting of night sweats, hot flashes, chills, sleep disturbances, insomnia, fatigue, irritability and inability to concentrate.

28. The method of claim 27, wherein the one or more symptoms associated with menopause are hot flashes or night sweats.

29. The method of claim 26, wherein the subject is peri-menopausal, menopausal, or post-menopausal.

30. The composition of claim 1, wherein the composition is obtained by a method comprising the steps of:
(i) placing the gum bases in a container with a sugar alcohol and heating to an internally-measured temperature of between 140-160° F. to melt the gum base;
(ii) combining the herbal extract comprising isoflavones, sweeteners and flavoring agents, and mixing;
(iii) adding the melted gum base mixture to the mixture from step (ii) and mixing;
(iv) conditioning the mixture from step (iii) for at least about 6 hours at a temperature less than or equal to 75° F. and a relative humidity of 60%;
(v) milling to form particulates;
(vi) mixing the particulates of step (v) with a lubricant; and
(vii) tableting the mixture from step (vi).
